(19) United States
(12) Patent Application Publication
(10) Pub. No.: US 2002/0150566 A1
(43) Pub. Date: Oct. 17, 2002

(54) Method of inhibiting cancerous cell proliferation using Ras mutants of GDP-bound conformation

(76) Inventors: Kun-Liang Guan, Ann Arbor, MI (US); Scott Stewart, Palo Alto, CA (US)

Correspondence Address: FLESHNER & KIM, LLP, P.O. Box 221200, Chantilly, VA, 20153-1200, US

(21) Appl. No.: 10/104,484

(22) Filed: Mar. 25, 2002

Related U.S. Application Data
(60) Provisional application No. 60/277,959, filed on Mar. 23, 2001.

(51) Int. Cl. A61K 38/51

(52) U.S. Cl. 424/94.5

(57) ABSTRACT

Methods for treating proliferative disorders, using GDP-bound Ras proteins, wild-type or mutant, such as RasN17N69, and A15N69 are described. Pharmaceutical compositions and kits for treatment of mammals, such as humans, with Ras-mediated proliferative disorders are also described.

### CLAIM TO PRIORITY

This non-provisional application claims priority to U.S. Provisional Application No. 60/277,959, the entire disclosure of which is incorporated herein by reference.

### STATEMENT TO RIGHTS TO INVENTIONS MADE UNDER FEDERALLY-SPONSORED RESEARCH AND DEVELOPMENT

The present invention was made with government support. Accordingly, the United States government has certain rights in the invention.

### BACKGROUND OF THE INVENTION

1. Field of the Invention

The present invention pertains to the field of Ras-mediated proliferative disorders in eukaryotic organisms, particularly animals.

2. Background of the Related Art

Normal cell proliferation is regulated by a balance between growth-promoting proto-oncogenes and growth-constraining tumor-suppressor genes. Tumerogenisis can be caused by genetic alterations to the genome that result in the mutation of those cellular elements that govern the interpretation of cellular signals, such as potentiation of proto-oncogene activity or inactivation of tumor suppression. It is believed that interpretation of these signals ultimately influences the growth and differentiation of a cell, and that misinterpretation of these signals can result in neoplastic growth (neoplasia).

Genetic alteration of the proto-oncogene Ras is believed to contribute to approximately 30% of all human tumors (Wiessmuller, L. and Wittinghofer, F. (1994), Cellular Signaling 6(3):247-267; Barbaci, M. (1987) A Rev. Biochem. 56,779-827). Activating mutations in Ras are found in most types of human malignancies, and are highly represented in pancreatic cancer (80%), sporadic colorectal carcinomas (40%-50%), human lung adenocarcinomas (15%-24%), thyroid tumors (50%), and myeloid leukemia (30%) (Millis, N E et al. (1995) Cancer Res. 55:1444; Chaubert, P et al. (1994), Am. J. Path. 144:767; Bos, J (1989) Cancer Res 49:4682).

Current methods of treatment for neoplasia include surgery, chemotherapy and radiation. Surgery is typically used as the primary treatment for early stages of cancer. However, many tumors cannot be completely removed by surgical means. In addition, metastatic growth of neoplasms may prevent complete cure of cancer by surgery. Chemotherapy involves administration of compounds having antitumor activity, such as alkylating agents, antimetabolites, and antitumor antibiotics. The efficacy of chemotherapy is often limited by severe side effects, including nausea and vomiting, bone marrow depression, renal damage, and central nervous system depression. Radiation therapy relies on the greater ability of normal cells, in contrast with neoplastic cells, to repair themselves after treatment with radiation. Radiotherapy cannot be used with many neoplasms, however, because of the sensitivity of the tissue surrounding the tumor. In addition, certain tumors have shown resistance to radiotherapy. In view of the drawbacks associated with the current means of treating neoplastic growth, the need still exists for improved methods for the treatment of most types of cancers.

### SUMMARY OF THE INVENTION

The invention is related to the discovery that the GDP forms of Ras, such as the GDP-bound Ras mutant, RasN17N69, can block oncogenic cellular transformation as well as inhibit oncogenic cell proliferation, and even reverse the tumorigenic activity of the oncogenic Ras.

Accordingly, the present inventions relates to a method for inhibiting cellular oncogenic transformation and proliferation, or reversing cellular oncogenic transformation in Ras-mediated neoplasia comprising administering to a mammal in need thereof an effective amount of a GDP-bound Ras protein. The Ras protein used in the present methods is preferably a non-interfering Ras and/or a membrane-associated Ras. The protein used in the present methods may be recombinantly made from an exogenous Ras gene carried on an expression vector or expressed from naturally occurring endogenous DNA or RNA. The protein used in the present method may be expressed in situ or it may be expressed in a host cell and isolated and purified before administration to the subject animal in need thereof. Because of the highly conserved nature of the Ras genes/proteins, it is intended to use Ras gene/protein of one species in another. The Ras protein used in the present methods may be a mutant or wild type Ras as long as it is GDP-bound.

In another embodiment of the present invention specific Ras mutants are described for use in the method of the present invention. These include but are not limited to RasN17N69 and RasA15N69.

In another preferred embodiment of the invention, a method is described for making a Ras mutant comprising mutating a RasN17 mutant in the switch II region. The switch II region preferably comprises the amino acid residues 62-70 of the Ras protein. In a more preferred embodiment of the invention, at least one of the residues 62-70 is mutated to alanine or serine.

An embodiment of the invention relates to the expression vectors encoding a GDP-bound Ras protein.

Yet, another embodiment of the present invention relates to pharmaceutical compositions and kits for use in the methods of the present invention. The pharmaceuticals of the present invention comprise a GDP-bound Ras protein or an expression vector expressing the same and a pharmaceutically acceptable carrier. The GDP-bound Ras of the pharmaceuticals of the present invention are preferably non-interfering Ras and/or membrane-associated Ras. Most preferred pharmaceuticals of the present invention comprise at least one of the two mutants: RasN17N69 and RasA15N69.

In a preferred embodiment of the invention, the Ras protein is administered by protein transduction. A preferred Ras protein in this embodiment is a fusion protein.

In another preferred embodiment of the invention, the present method is administered in conjunction with other anti-neoplastic treatments, such as radiation, chemotherapy, or surgical removal of at least part of the neoplasm.

The pharmaceuticals, kits, and methods of the present invention are intended for use in the treatment of a Ras-mediated disorder such as but not limited to neoplasia, particularly pancreatic cancer, sporadic colorectal carcinomas, lung adenocarcinomas, thyroid cancer, and myeloid leukemia.

The animals treated by the pharmaceuticals and methods of the present invention may be but not limited to a mammal such as cats, dogs, rabbits, mice, sheep, goat, cattle, horses, humans, and non-human primates.

### BRIEF DESCRIPTION OF THE DRAWINGS

The invention will be described in detail with reference to the following drawings:

FIG. 1. Inhibition of TCF mediated transcription by GDP-Ras mutants. A. PMA stimulated Gal4-ElkC activity is blocked by N17Ras expression. CV-1 cells were transfected with expression vectors for Gal4-ElkC, Gal4-luciferase and the indicated amount of N17Ras or a dominant negative MEK1 mutant, dnMEK1. Serum starved cells were stimulated for 8 hours with PMA (100 ng/ml) before harvesting and determination of luciferase activity. B. Dominant negative Ras isoforms, but not N17Rap1b, inhibits Gal4-ElkC activity. CV-1 cells were transfected with reporters as in panel A in the presence of either N17HRas, N17KRas, N17NRas, or N17Rap1b. Luciferase activity was determined 8 hours following PMA stimulation as in A. C. TCF, but not AP-1 or NF-kB, dependent transcription is inhibited by N17Ras. CV-1 cells were co-transfected with either c-fos, AP-1, or NF-kB luciferase reporter plasmids in the presence (hatched bars) or absence (solid bars) of N17Ras. Following an 8 hour stimulation with PMA as in panel A, luciferase activity was determined. All luciferase activity was normalized to a co-transfected b-galactosidase expression vector. Shown are representative examples from at least three independent experiments performed in duplicate.

FIG. 2. Elk-1 phosphorylation, but not ERK1 or RSK1 activity is inhibited by N17Ras expression. A. EGF, but not PMA, stimulated HA-ERK1 activity is inhibited by N17Ras expression. COS-1 cells were co-transfected with HA-tagged ERK1 together with either N17Ras, dnMEK1 or vector. Cells were either left untreated (solid bars) or stimulated for 5 minutes with EGF (50 ng/ml, light hatched bars) or PMA (100 ng/ml, dark hatched bars). HA-ERK1 activity was determined by an immune-complex kinase assay using MBP as a substrate (upper panel). Lane 0 denotes transfection control without HA-ERK1. A portion of each kinase reaction was blotted and probed with a-ERK antibody (lower panel). B. EGF, but not PMA, stimulated HA-ERK1 phosphorylation is blocked by N17Ras expression. COS-1 cells were co-transfected with HA-ERK1 and N17Ras or vector as in panel A. Quiescent cells were stimulated with either PMA or EGF for 5 minutes and harvested. Whole cell extracts were subjected to SDS-PAGE and immunoblotting with a-ERK (lower panel) of a-pERK (upper panel). The two lower bands detected by ERK antibodies are due to endogenous ERK1 and ERK2. C. Extended time course of ERK activation. COS-1 cells were transfected as in panel A. After serum starvation, cells were stimulated with PMA for the indicated times, followed by lysis and immunoblotting. D. PMA stimulated HA-RSK1 activity is not altered by N17Ras. COS-1 cells were transfected with HA-tagged RSK1 in the presence or absence of N17Ras, dnMEK1 or vector. Serum-deprived cells were stimulated with PMA for 20 minutes. HA-RSK1 kinase activity was determined by an immune-complex kinase assay (upper panel). A portion of each kinase reaction was blotted and probed with a-HA (lower panel). Shown for each are representative examples of at least three independent experiments. E. N17Ras expression blocks PMA stimulated Elk-1 phosphorylation at serine 383. COS-1 cells were co-transfected with expression vectors for Elk-1 and either vector, N17 or N17S186HRas. Cells were stimulated with PMA for 5 minutes and extracts were blotted and probed with a-Elk-1 (middle panel), a-phospho-Elk-1 (upper panel) and a-HRas (lower panel) as indicated.

FIG. 3. Inhibition of Elk-1 phosphorylation by Ras correlates with the ability to assume a GDP-bound conformation, but not the ability to inhibit endogenous Ras activation. A. Dominant interfering GDP-, but not GTP-, bound Ras inhibits MEK/ERK induced Elk-1 phosphorylation. COS-1 cells were transfected with active MEK1*, HA-ERK1 and Elk-1 together with either N17Ras or L61S186Ras. Cell lysates were blotted with various antibodies as indicated on the right side of the panel. L61S186Ras does not inhibit MEK/ERK-induced Elk-1 phosphorylation (lane 5). B. Selective inhibition of MEK3/p38 induced Gal4-ElkC, but not Gal4-ATF2, activity by N17Ras. CV-1 cells were co-transfected with expression vectors for constitutively active MEK3, MEK3-DE, and the indicated amount of p38, together with Gal4-luciferase and either Gal4-ElkC (solid bars) or Gal4-ATF2 (hatched bars). N17Ras or vector was included as indicated. C. Expression of N17N69Ras inhibits Elk-1 phosphorylation, but not endogenous Ras, activation. COS-1 cells were co-transfected with Elk-1 and HA-ERK1 with either N17Ras or N17N69Ras. Whole cell extracts were subjected to immunoblotting with the indicated antibodies.

FIG. 4. Oncogenic Ras induced transformation and transcriptional activation are inhibited by N17Ras. A. V12Ras-induced SRE, and c-fos promoter activity is blocked by GDP-Ras mutants. NIH3T3 cells were co-transfected with either SRE or c-fos luciferase constructs in the presence V12Ras. Where indicated, Ras mutants, HVH-1 or vector was included. B. Inhibition of V12Ras-induced Gal4-ElkC, Gal4-Sap1C, and Gal4-Sap2C activity by GDP-Ras mutants. NIH3T3 cells were transfected with Gal4-luciferase and the indicated Gal4 chimera together with the indicated Ras expression vector. All luciferase activity was normalized to a co-transfected b-galactosidase activity. Shown are representative examples from at least four independent experiments performed in duplicate. C. N17Ras blocks V12Ras induced focus-formation in NIH3T3 cells. Low passage NIH3T3 cells were transfected with the indicated Ras expression vectors in duplicate. 14 days post-transfection, foci were stained with crystal violet and scored. Shown is one of three independent experiments that yielded very similar results.

FIG. 5. N17Ras interferes with wild-type, but not oncogenic, Ras. A. N17Ras does not effect V12Ras GTP loading. COS-1 cells were transfected as indicated, followed by serum starvation and 32-PO4 labeling. Where indicated cells stimulated with EGF (50 ng/ml, Calbiochem) prior to immunoprecipitation with a-HA. Guanine nucleotides bound to the HA-tagged Ras were eluted and separated by TLC followed by autoradiography (left panel). Identically transfected cells were harvested for immunoblotting with a-HRas (upper right panel) or a-FLAG (lower right panel). B. N17Ras does not affect V12Ras-GST-RBD binding. COS-1 cells were transfected as indicated. After serum starvation, lysates were prepared and precipitated with GST-RBD and glutathione-sepharose. Eluted proteins were separated by SDS-PAGE and immunoblotted with a-HA (left panel). Additionally, a portion of the lysate was immunoblotted with a-HRas (right panel). C. Nucleotide binding status of HRas mutants. Cells were transfected with the indicated Ras expression vector. 48 hours post-transfection, cells were labeled with 32-PO4 for 4 hours followed by immunoprecipitation with a-HRas antibody. Bound nucleotides were eluted and subjected to TLC as in A (left panel). A portion of the immunoprecipitates were immunoblotted with a-HRas (right panel).

FIG. 6. Upper panel depicts the nucleotide sequence (SEQ ID NO 1) and the lower panel depicts the amino acid sequence (SEQ ID NO 2) of the GDP-bound Ras mutant, RasN17N69.

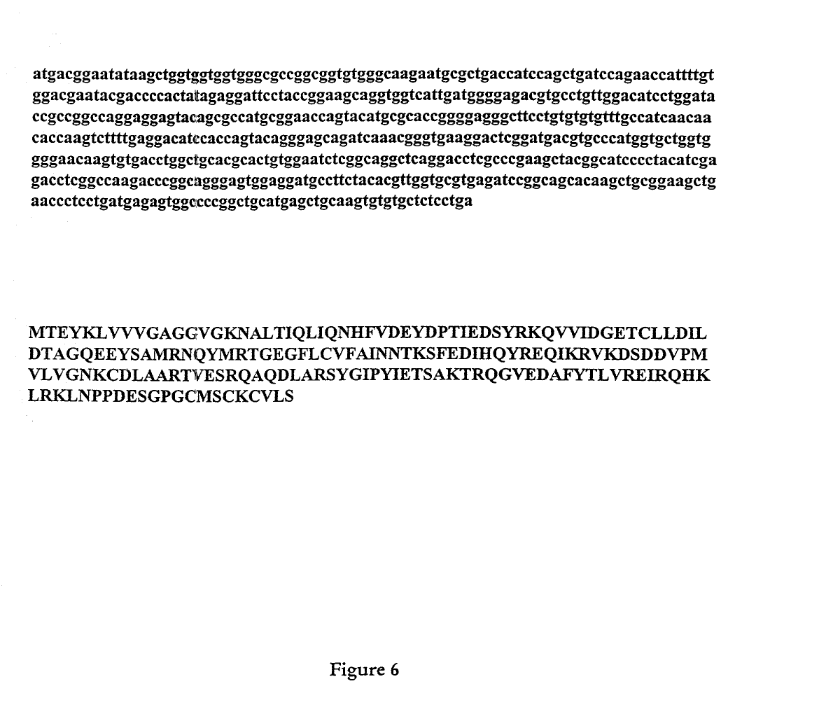

FIG. 7. Upper panel depicts the nucleotide sequence (SEQ ID NO 3) and the lower panel depicts the amino acid sequence (SEQ ID NO 4) of the wild type Ras mutant, RasN17N69.

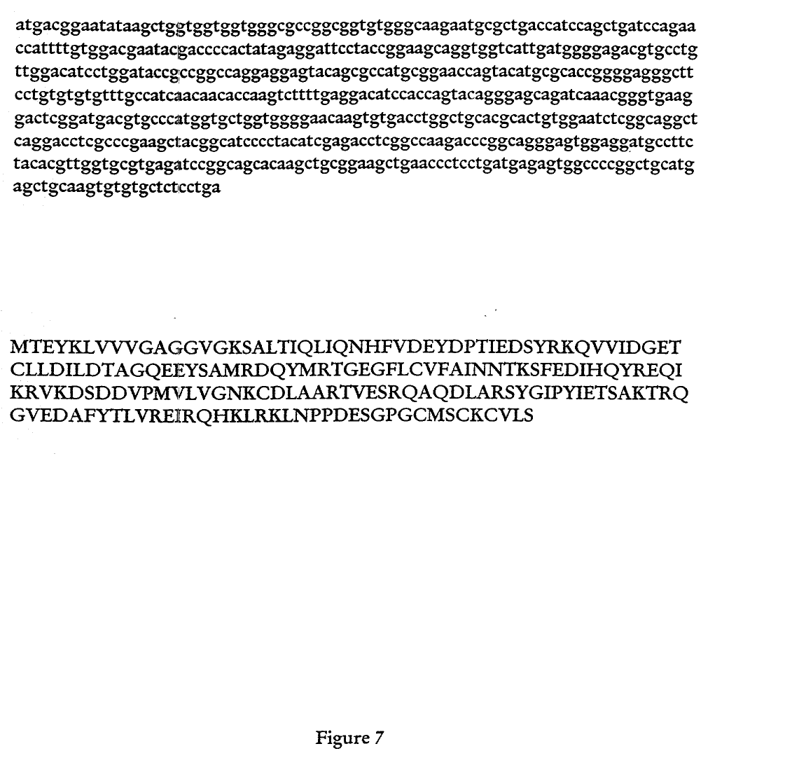

### DETAILED DESCRIPTION OF PREFERRED EMBODIMENTS

The following abbreviations are used throughout the specification: GTPase, guanine nucleotide triphosphate phosphatase; GST, glutathione S-transferase; EGF, epidermal growth factor; ERK1 , extra-cellular signal regulated protein kinase 1 or mitogen-activated protein kinase 1; MAP kinase, mitogen-activated protein kinase; MEK1 , mitogen-activated protein kinase kinase 1; HA, hemagglutinin epitope; GEF, guanine-nucleotide exchange factor; PMA, phorbol myristate acetate; SRE, serum response element; SRF, serum responsive factor; SOS, son-of-sevenless; TCF, ternary complex factor; p90 RSK1, 90 kDa ribosomal S6 kinase 1.

Ras-family GTPases cycle between inactive GDP- and active GTP-bound states. Oncogenic activation stabilizes Ras in a GTP-bound form, which is therefore constitutively active. Approximately 30 percent of all human tumors contain activating mutations in one of three Ras genes (H, K and NRas) (1,2). Expression of active Ras mutants in established cell lines can lead to cellular transformation, and, the same mutants cooperate with the myc oncoprotein to transform primary cells, demonstrating a key role for Ras in cellular transformation (3). Genetic studies in Drosophila and C. elegans have established that Ras plays critical roles in several developmental events, including photoreceptor differentiation and vulval development (2). Furthermore, microinjection of neutralizing Ras antibodies or expression of dominant negative Ras mutants demonstrated that Ras function is required for cell proliferation in response to serum and growth factors (4,5).

GTP-Ras; has been shown to physically interact with numerous downstream targets and to activate several different signaling pathways (1,2). One of the best characterized Ras-activated pathways is the Raf-MEK-ERK pathway, also known as the mitogen activated protein (MAP) kinase cascade (6). Ras directly binds Raf in a GTP dependent manner and this interaction appears to be critical for the activation of Raf. Activated Raf phosphorylates and activates MEK, which in turn phosphorylates and activates the MAP kinase, ERK. Activation of ERK is essential for numerous Ras-induced cellular responses including transcription activation of immediate early genes, such as c-fos (6-9).

The promoter of the proto-oncogene c-fos has been extensively characterized and is now considered a paradigm of transcription regulation in response to extra-cellular signals, including serum (7,9). The serum response element (SRE) within the c-fos promoter confers serum responsiveness to a basal promoter and functions via a transcription factor complex consisting of a dimeric serum response factor (SRF) and, in some cases, an associated ternary complex factor (TCF) family member (7,9). One well characterized member of the TCF family is a ubiquitously expressed 62 kD protein, Elk-1. MAP kinases phosphorylate numerous serine and threonine residues in the C-terminal transactivation domain of Elk-1, and in doing so, increases its transactivation potential (10-15). TCFs are thought to play significant roles in the induction of c-fos in response to oncogenic Ras and a variety of growth factors and cytokines (10,11,16-18). Thus, phosphorylation of TCFs by activated MAP kinases reveals a linear pathway from Ras activation to transcriptional regulation.

N17Ras is a dominant negative Ras mutant that binds GDP with preferential affinity over GTP. This property allows N17Ras to inhibit endogenous Ras activation by sequestering Ras-GEFs (5,19-26). Expression of N17Ras can effectively inhibit serum-dependent cell proliferation and this effect can be reversed by co-expression of oncogenic Ras or Ras-GEFs (5,20,21). Therefore, N17Ras has been proposed to selectively inhibit wild-type, but not oncogenic, Ras (23). In contrast to N17Ras, L61S186 is a cytoplasmic, GTP-bound interfering Ras mutant (23,27). L61S186Ras interferes with signaling via a mechanism that is likely to involve titration of effectors away from the endogenous, membrane associated Ras. Consistent with this, L61S186Ras appears to block signaling from both wild-type and oncogenic Ras (23). These observations suggest that N17Ras should always be recessive to V12Ras.

However, the dogma that the active GTP-bound form of Ras is dominant with respect to GDP-Ras is somewhat perplexing. For instance, a single copy of an active HRas gene, in the presence of a single copy of a wild type HRas gene, is not sufficient to transform Rat-1 cells (28). Furthermore, loss of a normal copy of Ras has been observed in numerous tumors containing active mutant Ras alleles (29-32). These observations suggest that the absence of normal Ras gene product may facilitate transformation by the remaining activated mutant Ras allele. Moreover, a GDP-bound Ras mutant, N17Ras, can effectively block transformation or neuronal survival induced by oncogenic Ras mutants (5,25).

The effects of expressing the dominant interfering Ras mutant, N17Ras, on growth factor and phorbol ester induced signaling were examined. Phosphorylation and activation of Elk-1, a well known MAP kinase substrate, in response to PMA was specifically inhibited by N17Ras expression. However, MAP kinase activity stimulated by phorbol esters was not affected by N17Ras. Expression of either N17Ras or A15Ras, another GDP-bound interfering mutant, inhibited Elk-1 activation induced by V12Ras. The ability of N17Ras to inhibit Elk-1 requires Ras membrane association. In contrast, the ability to inhibit Ras activation is not required for N17Ras to inhibit Elk-1 since N17N69Ras, a non-interfering GDP-bound Ras mutant, retains the ability to block Elk-1. Furthermore, it was observed that focus formation in NIH3T3 cells induced by GTP-Ras (V12Ras) is inhibited by N17Ras although it does not affect the nucleotide loading of V12Ras. These observations suggest that N17Ras may have functions in addition to interfering with endogenous Ras activation.

Discussion of Experimental Results

Experiments using Ras mutants have been instrumental in elucidating biological and biochemical functions of Ras. N17Ras is extensively used as a dominant negative mutant to probe Ras function since it interferes with Ras activation in vivo by formation of non-productive complexes with exchange factors (5,19-26). In fact, overexpression of N17Ras is usually the sole indicator for determining whether a particular signaling event involves Ras activation. The analogous mutant versions of Ras related GTPases, such as Rac, Rho, and CDC42, also act as dominant interfering mutants and are frequently utilized to determine roles for GTPases in signaling. However, to ascertain the involvement of a small GTPase in a given signaling pathway, it is essential to understand the mechanism of function of such dominant interfering mutants. It is reported herein a novel effect induced by dominant negative N17Ras, in addition to its ability to block Ras activation. The present results demonstrate that expression of N17Ras can inhibit Elk-1 activation independently of blocking endogenous Ras activation. These observations suggest that caution should be taken in interpreting data that rely upon dominant negative mutants to implicate a small GTPase in signaling.

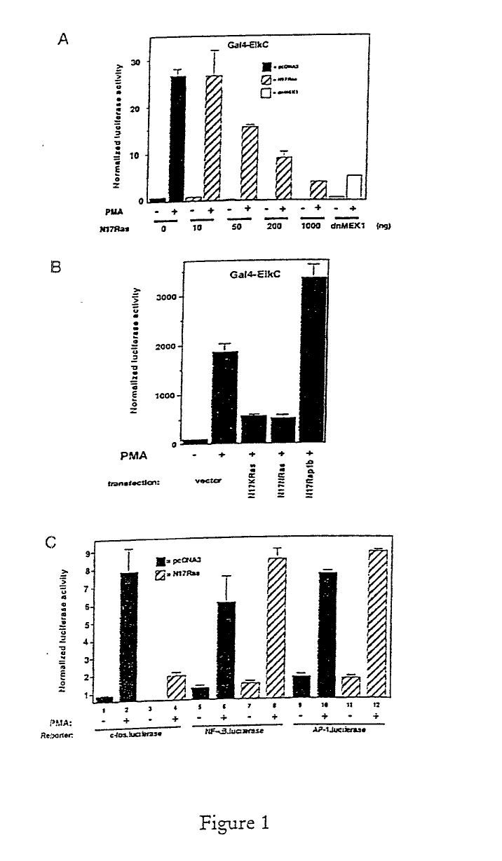

Expression of N17Ras alone can effectively inhibit serum dependent cell proliferation and this effect can be reversed by co-expression of oncogenic Ras or Ras-GEFs (5,20,21). It has therefore been assumed that the only function of N17Ras is to inhibit Ras activation. The data presented here, however, suggest that N17Ras may have functions besides inhibiting Ras activation. This argument is based on several observations. First, N17Ras expression can negatively regulate Elk-1 (FIGS. 1, 2E, 3, 4), a substrate of MAP kinases, yet have no effect on the activity of MAP kinase itself (40-42, 48). Second, N17Ras expression inhibits active MEK3-p38 induced Elk-1 activity as well as active MEK1 induced Elk-1 phosphorylation. This result is surprising since no GTP-dependent Ras function has been identified that regulates the direct activation of a MAP kinase by a MAP kinase kinase. Third, the abilities of two different classes of dominant negative Ras mutants to inhibit MEK-induced Elk-1 phosphorylation were compared. It was found that only the GDP-bound N17, but not the GTP-bound L61S186 mutant, inhibited Elk-1 in response to constitutively active MEK expression. Fourth, N17N69Ras also inhibits Elk-1 phosphorylation and oncogenic transformation, yet it neither inhibits cell growth (20,21) nor ERK activation. Furthermore, membrane association appears important for N17Ras function since a cytosolic mutant, N17S186Ras, can no longer inhibit either Elk-1 or transfomation. Lastly, N17Ras expression inhibits TCF activity induced by V12Ras, which is not subject to negative regulation by N17Ras.

The observations presented here can be explained by at least two hypothetical models: 1) An unidentified GTP-dependent Ras function, which is required for Elk-1 activation, is inhibited by N17Ras. This would explain why inhibition of Elk-1, but not ERK1, is observed. However, at the moment it is difficult to hypothesize a role for this unknown Ras effector in Elk-1 regulation. Furthermore, it cannot be clearly explained how a particular GTP-dependent Ras function, such as Elk-1 activation, could be inhibited by N17Ras while another, such as ERK activation, would not be affected in the same cells. One possibility is that distinct intra-cellular pools of Ras may be inhibited by N17Ras expression. For example, different pools of Ras, each regulated by distinct Ras-GEFs, may participate in ERK activation and Elk-1 activation, respectively. 2) N17Ras may regulate, directly or indirectly, the activity of an unidentified component(s) involved in Elk-1 regulation. Since GEFs are the only known targets for N17Ras, it is possible that N17Ras may regulate another GEF for a small GTPase.

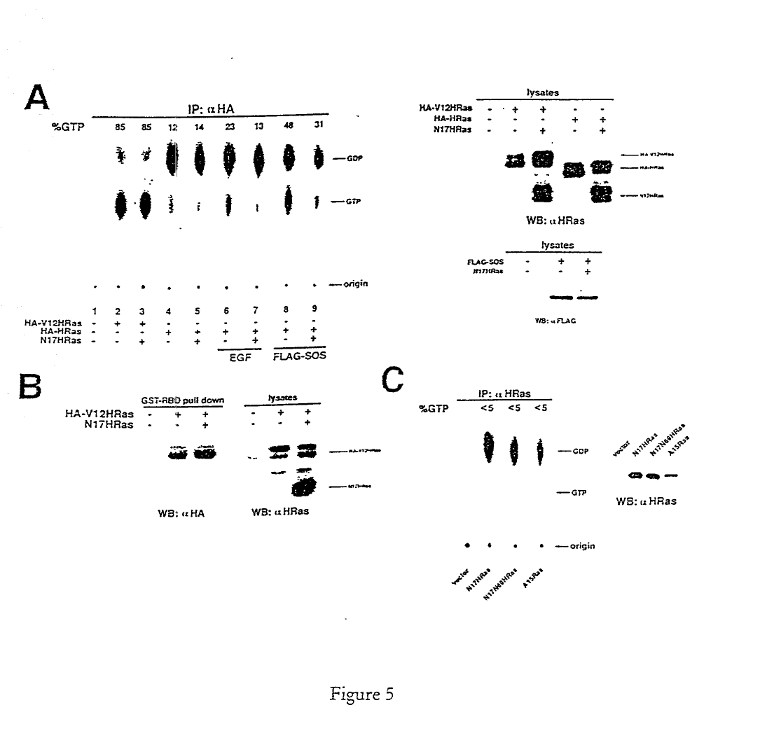

In vitro, N17Ras displays reduced nucleotide binding to both GDP and GTP, though the latter is much more severe (5,25,26). In vivo, the nucleotide binding status of N17Ras has not been examined. The results from in vivo labeling and immunoprecipitation experiments, carried out by the inventors, directly confirm that this N17Ras is mainly GDP-bound in vivo (FIG. 5C). Although there are no known GDP-dependent targets of Ras, inhibition of Elk-1 and transformation may be physiological function of GDP-Ras, since N17Ras is constitutively GDP-bound under physiological conditions. Furthermore, A15Ras which is also GDP-bound in vivo displays functions similar to N17Ras. Therefore, GDP-Ras itself may signal to an unknown effector molecule that leads to Elk-1 inhibition and suppression of transformation. Interestingly, recent evidence indicates that a Ras related GTPase, Bud1, which functions in bud site selection in yeast, directly interacts with one of its targets in a GDP-dependent manner (49). In addition, similar phenomena have been observed for another small GTPase, Ran1, which regulates nuclear protein transport (50). These examples provide direct evidence for a GDP-bound form of small GTPase in signaling.

The present observations have relied upon transfected GDP-bound Ras mutants. Although it may be intrinsically difficult to demonstrate that endogenous GDP-Ras functions in signaling in types of experiments presented here, studies of cancer progression provide genetic evidence that endogenous wild-type Ras, which is primarily GDP-bound, participates in suppressing the oncogenic potential of active Ras alleles. For, example, Bremner and Balmain have observed that loss of wild type, but not active HRas alleles, occurs at high frequencies during skin tumor progression in mice. Loss of wild-type HRas was frequently observed in tumors that harbor an activated HRas allele. Thus, amplification of active HRas alleles and/or loss of the wild-type copy of HRas appears to be consistent features of skin tumor development in mice (31). Similar results have been observed with NRas in mouse thymic lymphomas (29), as well as with K and NRas in clonal murine lymphoma (30), and with HRas in human cervical cancers (32). It is also interesting to note that a single copy of active Ras is not dominant with respect to a single copy of wild-type Ras in transforming the Rat-1 fibroblast cell line (28). Furthermore, the spontaneously transformed cells that arose from this V12Ras/Ras heterozygous cell line were found to contain either amplification of the active Ras allele or deletion of the wild-type copy (28). These observations suggest that wild type Ras, which is mainly in a GDP-bound form, may have an inhibitory effect on oncogenic transformation in the presence of active Ras alleles.

Other investigators have raised questions concerning the mechanism of N17Ras function in vivo. For example, it has recently been demonstrated that GTP-Ras dependent functions, such as c-Raf activation, are inhibited by neutralizing Ras antibody injection, but not by N17Ras expression (40). In addition, N17Ras has also previously been reported to inhibit cellular transformation induced by V12Ras (5), v-Raf induced transcription activation of the T-cell receptor b gene (51), and V12Ras induced neuronal survival (25), suggesting that GDP- and GTP-Ras may be co-dominant in some cases.

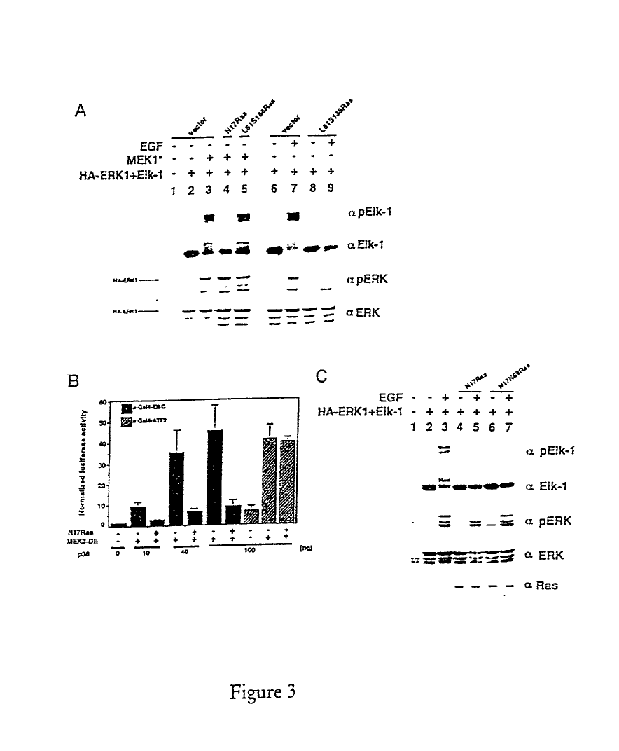

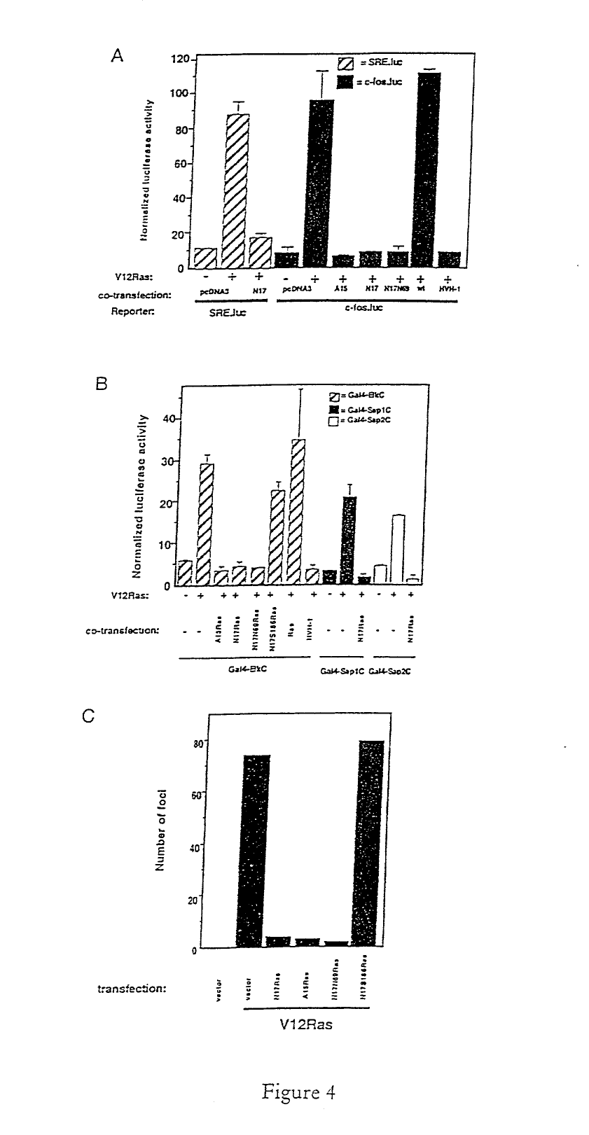

The data presented here suggest that caution must be taken when interpreting data that rely upon N17Ras as the sole means of implicating Ras function in signaling. This notion is certainly underscored by the fact that mutant Ras proteins, such as N17N69, which poorly interfere with EGF stimulated Ras activation (FIG. 3B) and cell growth (20,21), inhibit Elk-1 activation (FIG. 1) and NIH3T3 transformation (FIG. 3B). In addition, N17N69Ras attenuates c-fos promoter activity as effectively as N17 or A15Ras (FIG. 4A, and B).

The present invention relates to the treatment of proliferative disorders. A “proliferative disorder” is any cellular disorder in which the cells proliferate more rapidly than normal tissue growth. Thus a “proliferating cell” is a cell that is proliferating more rapidly than normal cells. The proliferative disorders include but are not limited to neoplasms. The neoplasms that may be treated with the methods of the present invention include solid tumors and hematopoietic neoplasms. A neoplasm is an abnormal tissue growth, generally forming a distinct mass, that grows by cellular proliferation more rapidly than normal tissue growth. Neoplasms show partial or total lack of structural organization and functional coordination with normal tissue. These can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoetic structures (structures pertaining to the formation of blood cells) including components of the immune system, are called leukemias and lymphomas. A tumor is the neoplastic growth of the disease cancer. As used herein, a “neoplasm,” also referred to as a “tumor,” is intended to encompass hematopoetic neoplasms as well as solid neoplasms. Other proliferative disorders include, but are not limited to, neurofibromatosis.

At least some of the cells of the proliferative disorder have a mutation in which the Ras gene (or an element of the Ras signaling pathway) is activated, either directly (e.g., by an activating mutation in Ras) or indirectly (e.g., by activation of an upstream element in the Ras pathway). Activation of an upstream element in the Ras pathway includes, for example, transformation with epidermal growth factor receptor (EGFR) or Sos. A proliferative disorder that results, at least in part, by the activation of Ras, an upstream element of Ras, or an element in the Ras signaling pathway is referred to herein as a “Ras-mediated proliferative disorder.”

One neoplasm that is particularly susceptible to treatment by the methods of the present invention is pancreatic cancer because of the prevalence of Ras-mediated neoplasms associated with pancreatic cancer. Other neoplasms that are particularly susceptible to the treatment by the methods of the present invention include sporadic colorectal carcinomas, lung adenocarcinomas, thyroid tumors, and myeloid leukemia. Additionally, it is intended that the present invention be used in the therapy of other types of cancer such as breast cancer, central nervous system cancer (e.g., neuroblastoma and glioblastoma), peripheral nervous system cancer, prostate cancer, renal cancer, adrenal cancer, liver cancer, and lymphoma.

Based on the results of the experiments discussed above, a method for treatment of proliferative disorders, including but not limited to cancer therapy, has been designed that relies on the demonstrated property of the GDP-bound Ras mutants to inhibit or block Ras oncogenic activity, inhibit proliferation of neoplastic cells, and even reverse cellular oncogenic transformation. Furthermore, the evidence indicates that GDP-bound wild type Ras has similar anti-neoplastic effect on Ras-mediated transformation. Therefore, it is intended that increased cellular levels of GDP-bound wild type Ras be used as a method for cancer therapy.

Therefore, a preferred embodiment of the invention relates to administration of GDP-bound Ras mutants and/or GDP-bound wild type Ras to an animal, particularly, a mammal, in need thereof.

The preferred Ras mutants for use in the methods of the present invention include but are not limited to the GDP-bound RasN17N69, and RasA15N69.

The preferred Ras mutants used in the present invention are associated with the cellular membrane, and referred to herein as membrane-associated Ras. It is also preferred that the Ras mutants used in the invention not interfere with endogenous Ras function, these mutants are referred to as non-interfering Ras mutants.

It is also intended that the methods of the present invention make use of the other members of the Ras super family small GTPases. Ras belongs to a large family of small GTPases. Based on structure and function, the Ras super family GTPases can be divided into several subgroups. They include but are not limited to Ras, Rho/Rac, Ran, Rab, and ARF. All of these GTPases share very similar biochemical properties: binding of GTP and GDP and hydrolysis of GTP. The discovery herein that a GDP-bound Ras mutant, which does not interfere with endogenous small GTPase activation, can specifically block the endogenous function of a specific GTPase can also be applied to the other members of the Ras super family of small GTPases. For example, mutants similar to RasN17N69 can also be constructed with other members of the Ras super family. Those mutants may have potential therapeutic applications to block the corresponding endogenous GTPase functions. Therefore, the various embodiments of the invention described herein may be practiced using such mutants of small GTPases.

Other mutants for use with the methods of the present invention may be made as follows. The switch II region (amino acid residues 60-72) of Ras is known to be involved in interaction with Ras Activator GEF. Therefor, other mutations in the switch II region could also result in a Ras mutant unable to interact with GEF. Combination of such switch II mutations with Ras N17 (known to interact with the Ras GEF SOS) will produce Ras proteins similar to RasN17N69. Such Ras mutants, containing N17 and switch II mutations, may be screened for (by methods including but not limited to exemplified in Example 1, below) their ability to reverse transformation.

A method for isolating useful mutants that contain mutations in the switch II region is (but not limited to) the following: Each individual amino acid in the switch II region (residues 62-70) will be mutated, using any of the variety of known methods in the art, to either alanine or serine in the RasN17 background. Ras N17, as mentioned above, is known to interact with the Ras GEF SOS. Interaction of each Ras N17 mutant with SOS will be determined by protein-protein interaction, using known methods in the art. Those mutants which have lost their ability to interact with SOS, will be further characterized for their ability to interface with endogenous Ras activation. The preferred RasN17 switch II mutants are those which do not block endogenous Ras action. Such mutants will be further screened for their activity to reverse the oncogenic phenotypes of cancer cells.

It is intended that any one of the naturally occurring or any forms of the recombinantly produced protein, fusion protein or otherwise, be used in the methods of the present invention. The wild type or mutant proteins, whether naturally occurring or recombinantly produced (including natural proteins expressed by targeted or random activation of endogenous gene expression), may be isolated and purified, using methods known in the art, such as any number of the conventional chromatography methods, such as affinity chromatography using tagged 6-hintidine or glutathione S-transferase. The recombinantly produced proteins may be engineered for expression in eukaryotic or prokaryotic cells. E. coli is a preferred and commonly used host for recombinant expression. However, the most preferred host cells can be determined in each given situation.

Administering the GDP-bound Ras protein of the invention to an animal, particularly a mammal, in need thereof indicates that the GDP-bound Ras protein is administered in a manner that it contacts the proliferating cells or cells of the neoplasm (the neoplastic cells). It is intended that such administration includes direct application of the protein, induction of a native gene encoding the protein, or introduction of an exogenous nucleic acid, such as but not limited to an expression vector that expresses the protein at the site of the targeted proliferating or neoplastic cells (in situ).

In the case of introducing an exogenous nucleic acid encoding the protein, such as an expression vector that expresses the protein in situ, the nucleic acid molecule preferably comprises the necessary regulatory sequences for transcription and/or translation in the cells of the animal.

The exogenous nucleic acid may be DNA or RNA. According to the present invention, the DNA or RNA that encodes the protein is introduced into the cells of an individual where it is expressed, thus producing the preferred GDP-bound Ras protein. The DNA or RNA encoding the desired protein is linked to regulatory elements necessary for expression in the cells of the individual. Regulatory elements for DNA expression include a promoter and a polyadenylation signal. In addition, other elements, such as Kozak region, may also be included in the genetic construct.

As used herein, the term “genetic construct” refers to the DNA or RNA molecule that comprises a nucleotide sequence which encodes the desired protein and which includes initiation and termination signals operably linked to regulatory elements including a promoter and polyadenylation signal capable of directing expression in the cells of the subject animal.

As used herein, the term “expressible form” refers to gene constructs which contain the necessary regulatory elements operably linked to a coding sequence that encodes a target protein, such that when present in the cell of the individual subject to treatment, the coding sequence will be expressed.

As described above, genetic construct comprise a nucleotide sequence that encodes the desired protein operably linked to regulatory elements needed for gene expression. Accordingly, incorporation of the DNA or RNA molecule into a living cell results in the expression of the DNA or RNA encoding the desired protein and thus, production of the desired protein.

When taken up by a cell, the genetic construct which includes the nucleotide sequence encoding the desired protein operably linked to the regulatory elements may remain present in the cell as a functioning extrachromosomal molecule or it may integrate into the cell's chromosomal DNA. DNA may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication.

The molecule that encodes the desired protein may be DNA or RNA which comprise a nucleotide sequence that encodes the desired protein. These molecules may be cDNA, genomic DNA, synthesized DNA or a hybrid thereof or an RNA molecule such as mRNA. Accordingly, as used herein, the terms “expression vector,” “genetic construct,” “gene construct,” “nucleic acid,” and “nucleotide sequence” are meant to refer to both DNA and RNA molecules.

The regulatory elements necessary for gene expression of a DNA molecule include: a promoter, an initiation codon, a stop codon, and a polyadenylation signal. In addition, enhancers are often required for gene expression. It is necessary that these elements be operably linked to the sequence that encodes the desired protein and that the regulatory elements are operable in the individual to whom they are administered.

Initiation codons and stop codons are generally considered to be part of a nucleotide sequence (if DNA) that encodes the desired protein. However, it is necessary that these elements are functional in the individual to whom the gene construct is administered. The initiation and termination codons must be in frame with the coding sequence.

Promoters and polyadenylation signals used must be functional within the cells of the individual subject to treatment.

Examples of useful promoters, other than those specifically exemplified herein, for the practice of the present invention include but are not limited to promoters from Simian Virus 40 (SV40), Mouse Mammary Tumor Virus (MMTV) promoter, Human Immunodeficiency Virus (HIV) such as HIV Long Terminal Repeat (LTR) promoter, Moloney virus, ALV, Cytomegalovirus (CMV) such as the CMV immediate early promoter, Epstein Bar Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention include but are not limited to SV40 polyadenylation signals, e.g. the SV40 polyadenylation signal which is in pCEP4 plasmid (Invitrogen, San Diego, Calif.) and LTR polyadenylation signals.

In addition to the regulatory elements required for DNA expression, other elements may be included in the DNA molecule. Such additional elements include enhancers. The enhancer may be selected from the group including but not limited to: human actin, human myosin, human hemoglobin, human muscle creatine and viral enhancers such as those from CMV, RSV and EBV.

Genetic constructs for use in mammals can be provided with mammalian origin of replication in order to maintain the construct extrachromosomally and produce multiple copies of the construct in the cell. Plasmids pCEP4 and pREP4 from Invitrogen (San Diego, Calif.) contain the Epstein Bar virus origin of replication and nuclear antigen EBNA-1 coding region which produces high copy episomal replication without integration.

In some preferred embodiments, the vector used is selected from those described in Example 1. In aspects of the invention relating to gene therapy, constructs with origins of replication including the necessary antigen for activation are preferred.

In order to maximize protein production, regulatory sequences may be selected which are well suited for gene expression in the target cells to which the construct is administered. Moreover, codons may be selected which are most efficiently transcribed in the cell. One having ordinary skill in the art can produce DNA constructs which are functional in the cells.

In order to test expression, genetic constructs can be tested for expression levels in vitro using tissue culture of the cells to be targeted for administration by the methods and pharmaceuticals of the present invention.

The route by which the GDP-bound Ras proteins of the invention are administered, as well as the formulation, carrier or vehicles, will depend on the location as well as the type of the neoplasm. A wide variety of administration routes can be employed. For example, for a solid neoplasm that is accessible, the GDP-bound Ras protein can be administered by injection directly into the neoplasm. For a hematopoetic neoplasm, for example, the GDP-bound Ras protein can be administered intravenously or intra vascularly. For neoplasms that are not easily accessible within the body, such as metastases or brain tumor, the GDP-bound Ras protein is administered in a manner that it can be transported systemically through the body of the subject animal and thereby reach the neoplasm (e.g. intrathecally, intravenously, intramuscularly). Alternatively, the GDP-bound Ras protein can be administered directly to a single solid neoplasm, where it then is carried systemically through the body to metastases. The GDP-bound Ras protein can also be administered subcutaneously, intraperitoneally, topically (e.g., for melanoma), orally (e.g., for oral or esophageal neoplasm), rectally (e.g., for colorectal neoplasm), vaginally (e.g., for cervical and vaginal neoplasm), nasally or by inhalation spray (e.g., for lung neoplasm).

An animal, particularly a mammal, in need of GDP-bound Ras protein therapy is one that may have a proliferative disorder or tumor or has been diagnosed with a proliferative disorder or tumor or has been previously diagnosed with a proliferative disorder or tumor, the tumor or substantially all of the tumor has been surgically removed and the subject animal is suspected of harboring some residual tumor cells.

Because the Ras protein structure and function is highly conserved in all eukaryotes, the methods of the present invention may be applied to a variety of eukaryotic organisms, such as but not limited to animals, particularly mammals, as well as other animals such as reptiles, fish, and bird. Hence, the approaches described herein for mammals are intended for appropriate modification and use in other eukaryotes, particularly animals. The mammals which may benefit from the methods of the present invention include but are not limited to felines, for example cat, canines, for example dog, horse, goat, cattle, sheep, pig, humans, and non-human primates.

Additionally, it is intended that Ras gene/protein from one eukaryotic species be administered to another eukaryotic species. Such cross species application is possible also because of the highly conserved nature of Ras genes/proteins.

A preferred method of administering the GDP-bound wild type or mutant Ras is by in situ expression of the gene. The in situ expression is carried out, preferably, by expressing an exogenously introduced GDP-bound wild type or mutant Ras. For this purpose a variety of expression vectors may be used, for example, see the materials and methods section of the Examples, below, and many described above.

Additionally, it is also intended and within the scope of the present invention to induce expression and/or up-regulation (increased expression) of endogenous genes by targeted or random activation of gene expression using a variety of known methods in the art. Random activation of gene expression (RAGE) is available through Athersys, Inc., Cleveland, Ohio. Methods for targeted alteration of endogenous DNA sequences are known in the art. See, for example, U.S. Pat. No. 6,200,812. Using homologous recombination technology, the endogenous wild type chromosomal Ras gene may be activated to express the protein in increased amounts. For example, additional enhancer sequences may be engineered into the endogenous sequences to up-regulate expression. A variety of techniques may be used to increase gene expression, and it is not intended to limit the present invention to any particular method.

Similarly, a variety of methods are known and available in the art for the delivery of the exogenous gene into the target cells. For a review of the various useful delivery systems see Advanced Drug Delivery Reviews, Vol. 44 (2000) pp. 3-21.

For example, the nucleic acid molecule comprising the Ras gene of the present invention may be administered to the animal in need thereof, alone, as naked polynucleotides, or in combination with substances that facilitate intact uptake of the nucleic acids into mammalian cells, e.g. urea (see U.S. Pat. No. 6,197,755), lipophilic cationic compounds, proteins, et cetera.

Preferably, the GDP-bound Ras gene is co-administered with an agent which enhances the uptake of the molecule by the cells. For example, it may be combined with a lipophilic cationic compound which may be in the form of liposomes. The use of liposomes to introduce nucleic acids into cells is known in the art. The following references, for example, describe the method in detail: U.S. Pat. Nos. 4,897,355 and 4,394,448, the disclosures of which are incorporated herein by reference in their entirety. See also U.S. Pat. Nos. 4,235,871, 4,231,877, 4,224,179, 4,753,788, 4,673,567, 4,247,411, 4,814,270 for general methods of preparing liposomes comprising biological materials.

Liposomes have been approved by FDA for gene transfer in humans. Using liposomes, plasmid DNA is transferred in liposomes directly to the target cell in situ. See Nable, E G et al. (1990) Science 249:1285-1288. A preferred cationic lipid delivery system

A preferred cationic lipid delivery system for transfecting cells with a plasmid containing the GDP-bound Ras gene is the commercially available Lipofectin. Lipofectin can efficiently deliver polyanionic plasmid DNA into the cytoplasm and nucleus of cells. See Behr, J. P. (1994) Bioconjugate Chem. 5, 382-389. Lipofectin is a 1:1 (wt/wt) liposome formulation of the cationic lipid N-[1-(2,3,-dioleyloxy)propyl]-N,N,N-trimethylammonium chloride (DOTMA) and dioleoylphosphatidylethanolamine (DOPE). An improved version of Lipofectin is the serum-resistant cytofectin, termed GS 2888 cytofectin. See Lewis, J. G. et al., PNAS USA 93:3176-3181 (1996). GS 2888 cytofectin efficiently transfects plasmid DNA into many cell types in the presence or absence of 10% serum, uses a 4- to 10-fold lower concentration of the agent as compared to Lipofectin, and is about 20-fold more effective in the presence of serum when compared to Lipeofectin.

In addition, the genetic constructs of the present invention may be conjugated to a peptide that is ingested by cells. Examples of useful peptides include peptide hormones, antigens or antibodies, and peptide toxins. By choosing a peptide that is selectively taken up by the target cells, specific delivery of the genetic construct may be effected.

A preferred method is by protein transduction. Several proteins can traverse biological membranes through protein transduction. Small sections of these proteins (10-16 residues long) are responsible for this. Linking these domains covalently to compounds, peptides, antisense peptide nucleic acids or 40-nm iron beads, or as in-frame fusions with full-length proteins, lets them enter any cell type in a receptor and transporter-independent fashion. Moreover, several of these fusions have been shown to deliver to all tissues, even crossing the blood-brain barrier. For a detailed description, see Schwartz, S R et al. (2000) Trends in Cell Biology 10(7):290-5; and Schwartz, S R et al. (1999) Science 285 (5433):1569-72.

Therefor, a form of the Ras protein used in the method of the present invention is a fusion protein. A variety of fusion proteins, other than ones described above, are intended. Fusion proteins used in the methods of the present invention may have been expressed recombinantly and later isolated and/or purified. The non-Ras portion of the fusion proteins may have been engineered in for a variety of reasons, including but not limited to secretion from the host cell, increased levels of expression, etc. Hence, a variety of fusion proteins and methods of making and using the same are intended and are known to the skilled artisans.

Other gene transfer methods include but are not limited to receptor-mediated transfer, wherein the genetic construct, e.g. plasmid DNA, is coupled to a peptide that binds a cell surface receptor. The plasmid DNA is thereby internalized into the cell and can be expressed thereafter. See, for example, U.S. Pat. No. 5,922,859. A similar technology takes advantage of the cells' ability for endocytosis. Nucleic acid of interest, e.g. the expression vectors of the present invention, having been coupled with an internalizing factor and an endosomolytic agent via polycationic polymers may be transferred into the cell cytoplasm. For example, see U.S. Pat. No. 6,077,663.

Viral vectors (retroviral and non-retroviral) are also considered to be premier vectors for gene transduction. Non-retroviral vectors include but are not limited to adenovirus, adeno-associated virus, herpes virus, mumps and poliovirus vectors.

Retrovial vectors have been used in approved gene transfer trials in humans. Retroviral vectors in this context are retroviruses from which all viral genes have been removed or altered so that no vial proteins are made in cells infected with the vector. Viral replication functions are provided by the use of retrovirus packaging cells that produce all of the viral proteins but that do not produce infectious virus. Introduction of the retroviral vector DNA into packaging cells results in production of virions that carry vector RNA and can infect target cells, but no further virus spread occurs after infection. In order to distinguish this process of gene transfer from a natural viral infection (where the virus continues to replicate and spread), often, the term transduction rather than infection is used.

The present invention also includes pharmaceutical compositions which contain, as the active ingredient, one or more of the GDP-bound proteins, whether wild-type or mutant, or expression vectors expressing the same, together with a pharmaceutically carrier or excipient. In making the compositions of the present invention, the active ingredient (the GDP-bound proteins of the present invention or expression vectors expressing the same) is usually mixed with an excipient, diluted with an excipient, or enclosed within such a carrier which can be in the form of a capsule, sachet paper or other container. When the pharmaceutically acceptable excipient acts as a diluent, it can be a solid, semi-solid, or liquid material, which acts as a vehicle, carrier or medium for the active ingredient.

Thus, the compositions can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose, dextrose, sucrose, sorbitol, manitol, starches, gum acacia, calcium phosphate, alginates, tragacanth, gelatin, calcium cilicate, microcrystalline cellulose, polyvinylpyrrolidone, cellulose, methyl cellulose, and sterile water. The formulations can additionally include lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principle active ingredient (the GDP-bound proteins of the present invention or expression vectors expressing the same) is mixed with a pharmaceutical excipient to form a solid preformulation composition containing a homogenous mixture of a compound of the present invention. When referring to these preformulation compositions as homogeneous, it is meant that the active ingredient is dispersed throughout the composition so that the composition may be readily subdivided into equally effective unit dosage forms such as tablets, pills and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former.

The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable acceptable excipients as described herein. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device, or the nebulizing device may be attached to a face mask, tent, or intermittent positive pressure breathing machine. Solutions, suspensions, or powder compositions may be administered, preferably orally or nasally, from devices which deliver the formulation in an appropriate manner.

Another preferred formulation employed in the methods of the present invention employs transdermal delivery devices (patches). Such transdermal patches may be used to provide continuous or discontinuous infusion of the active ingredient (GDP-bound proteins of the present invention, whether wild-type or mutant, or expression vectors expressing the same) in controlled amounts. The construction and use of transdermal patches for the delivery of pharmaceutical agents is well known in the art. See, for example, U.S. Pat. No. 5,023,252, herein incorporated by reference. Such patches may be constructed for continuous, pulsatile, or on demand delivery of the pharmaceutical agents.

Other suitable formulations for use in the present invention can be found in Remington's Pharmaceutical Sciences.

The GDP-bound proteins of the present invention or the expression vectors expressing the same, or the pharmaceutical compositions comprising the GDP-bound proteins of the present invention or the expression vectors expressing the same, may be packaged into convenient kits providing the necessary materials packaged in suitable containers. It is contemplated the kits may also include chemotherapeutic agents.

The GDP-bound proteins of the present invention or the expression vectors expressing the same are administered in an amount that is sufficient to treat the proliferative disorder (e.g. an “effective amount”). A proliferative disorder is “treated” when administration of GDP-bound proteins of the present invention or the expression vectors expressing the same to the cells results in inhibition of oncogenic transformation or cell proliferation, or reversal of cellular oncogenic transformation. This may result in a reduction in size of the neoplasm, or in a complete elimination of the neoplasm. Preferably the effective amount is that amount able to inhibit tumor cell growth. For example, the effective amount may be from about 15-100 mg of RasN17N69 for an individual with 60 kg body weight. The effective amount will be determined on individual basis and may be based, at least in part, on consideration of: whether a GDP-bound protein of the invention or an expression vector expressing the same is being administered; the chosen route of administration; the chosen vehicle for cellular delivery of the protein or the expression vector; the individual's size, age, sex; the severity of the patient's symptoms; the size and other characteristics of the neoplasm; and the like. The course of therapy may last from several days to several months or until diminution of the disease is achieved.

The GDP-bound proteins of the present invention or the expression vectors expressing the same can be administered in a single dosage, or multiple doses (i.e., more than one dose). The multiple doses can be administered concurrently, or consecutively (e.g. over a period of days or weeks). GDP-bound proteins of the present invention or the expression vectors expressing the same can be administered to more than one neoplasm or site in the same individual.

It is contemplated that GDP-bound proteins of the present invention or the expression vectors expressing the same may be administered in conjunction with surgery or removal of the neoplasm. Therefore, provided herewith are methods for the treatment of a solid neoplasm comprising surgical removal of the neoplasm and administration of the GDP-bound proteins of the present invention or the expression vectors expressing the same at or near to the site of the neoplasm.

It is contemplated that the GDP-bound proteins of the present invention or the expression vectors expressing the same may be administered in conjunction with or in addition to radiation therapy.

It is contemplated that the GDP-bound proteins of the present invention or the expression vectors expressing the same may be administered in conjunction with or in addition to other anticancer compounds or chemotherapeutic agents. Chemotherapeutic agents are compounds which may inhibit the growth of tumors. Such agents include, but are not limited to, 5-fluorouracil, mitmycin C, methotrxate, hydroxyurea, cyclophosphomide, dacarbazine, mitoxantrone, anthrachclins (Epirubicin and Doxurubicin), antibodies to receptors, such as herceptin, atopside, pregnasome, platinum compounds such as carboplatin and cisplatin, taxanes such as taxol and taxotere, hormone therapies such as tamoxifen and anti-estrogens, interferons, aromatase inhibitors, progestational agents and LHRH analogs.

It is contemplated that the GDP-bound proteins of the present invention or the expression vectors expressing the same may be administered to metastatic tumors. Therefore, in an embodiment of the invention, a method is provided for reducing the growth of metastatic tumors in a mammal comprising administering to the mammal an effective amount of the GDP-bound proteins of the present invention or the expression vectors expressing the same.

The various embodiments and advantages are merely exemplary and are not to be construed as limiting the present invention. The description of the present invention is intended to be illustrative, and not to limit the scope of the claims. Many alternatives, modifications, and variations will be apparent to those skilled in the art.

### EXAMPLES

Materials and Methods

In carrying out the experiments discussed below, the following materials and methods were used.

Cell culture and transfection. COS-1 and CV-1 cells were grown in Dulbecco's modified Eagle's medium (DMEM) containing 10% fetal bovine serum (FBS, Life Technologies). NIH3T3 cells were grown in DMEM containing 10% calf serum (Life Technologies). Transfections were performed using either DEAE-dextran as described previously (33) or LIPOFECTAMINE (Life Technologies) as recommended by the manufacturer.

Plasmid construction. Expression vectors encoding Ras mutants were constructed by amplifying the appropriate mutant cDNA (templates encoding mutant Ha-Ras cDNAs were generously provided by Dr. L. Quilliam, Indiana University) by polymerase chain reaction (PCR) followed by subcloning them into the mammalian expression vector pcDNA3.1 (Invitrogen) or pcDNA3-HA (33). The identities of all constructs were confirmed by DNA sequencing. Expression vectors encoding Elk-1, HA-ERK1, active MEK1 (MEK1*), active MEK3 (MEK3-DE), p38 MAP kinase, and V12Ras have been described (33). Expression vectors for SOS (34) (pEF-Flag-SOS), HA-RSK1 (35) (pMT2-HA-RSK1), N17Rap1b (pcDNA3-N17Rap1b) were kindly provided by Drs. J. Pessin (University of Iowa), Y. Zhao (University of Michigan), and P. Stork (Oregon Health Sciences University), respectively.

Luciferase assays. In general, CV-1 or NIH3T3 cells in 3.5 cm wells were transfected with 50 ng of Gal4 TCF chimeras (10,18) or Gal4-ATF2 (36), 100 ng of a 5×Gal4-luciferase reporter, and 100-250 ng each expression vector. c-fos luciferase (16), 4×AP-1 luciferase (Stratagene) and 5×NF-kB luciferase (Stratagene) reporter genes were typically used at a concentration of 0.25 μg/3.5 cm well. Total DNA was kept constant by the addition of the appropriate amount of pcDNA3.1 for all transfections. Luciferase assays were performed as described previously (33) and normalized for transfection efficiency and using a co-transfected b-galactosidase expression vector.

Immunoblotting. Whole cell extracts were separated by SDS-PAGE, transferred to PVDF membranes (Millipore) and blotted with the indicated antibodies according to standard methods. a-ERK has been described (37); a-active MAP kinase was purchased from Promega; a-Elk-1 and a-phospho-383 Elk-1 were purchased from New England Biolabs; a-HA was purchased from Babco; a-Flag was purchased from Sigma; a-HRas C-20 was purchased from Santa Cruz Biotechnology.

Kinase assays. ERK kinase assays were performed as described previously (33). For RSK1 kinase assays, HA-tagged RSK1 was transfected into COS-1 cells as indicated in the figure legend and immuinoprecipitated with a-HA antibody. Immunoprecipitated RSK1 activity was assayed using the S6 Kinase Assay Kit (Upstate Biotechnology) and quantified by scintillation counting.

Guanine nucleotide binding determination. Thirty-six hours post transfection, cells were metabolically labeled with 0.5 mCi/ml 32PO4 for 4 hours. Cells were solublized in buffer (1% TritonX-100, 50 mM HEPES-KOH pH 7.5, 150 mM NaCl, 5 mM MgCl2 containing a cocktail of protease and phosphatase inhibitors). Ras was immunoprecipitated with a-Ras C-20 antibody or a-HA, followed by extensive washing in lysis buffer, and elution of bound nucleotides. Determination of the ratio of GDP versus GTP bound to each Ras mutant was performed as described (38).

NIH3T3 cell focus formation assays. Focus formation assays were performed essentially as described previously (39). Briefly, low passage NIH3T3 were transfected with 50 ng V12Ras with various Ras mutants (0.5 μg, except N17N69Ras, 1 μg). 24 hours post transfection, cells were trypsinized and plated in 10 cm dishes. Cells were maintained in DMEM containing 10% calf serum and antibiotics. Fresh media was added every four days. 14 days post-transfection foci were stained with methylene blue. Ras transformed morphology was examined under a light microscope (39).

### Example 1

The following experimental results were obtained:

N17Ras specifically inhibits TCF dependent transcription. To more clearly understand the functions of Ras in MAP kinase mediated transcriptional activation, the dominant interfering Ras mutant, N17Ras (5), was tested on Gal4-ElkC activity in CV-1 and COS-1 cells. In these cell lines PMA stimulated ERK activity is not affected by N17Ras expression (40-43). It was observed that expression of N17Ras effectively blocked reporter gene activity, both under based and PMA stimulated conditions, in a dose dependent manner (FIG. 1A, hatched bars). In addition, expression of a dominant negative version of the ERK activator MEK1, dnMEK (44), also significantly decreased normalized Gal4-ElkC activity (FIG. 1A, open bars). In addition, N17KRas and N17NRas, dominant interfering versions of the two other human Ras isoforms were tested. Like HRas, expression of increasing amounts of dominant negative K or NRas inhibited PMA-induced Gal4-ElkC activity. However, expression of N17Rap1b, a closely related small GTPase did not inhibit Gal4-ElkC activity (FIG. 1B), suggesting that the inhibitory effect observed on Elk-1 may be specific to dominant negative Ras mutants. These results suggest that both Ras and MAP kinase activation are required for Gal4-ElkC activity in response to PMA.

Similar co-transfection experiments were performed using a c-fos promoter reporter gene which requires intact TCF DNA binding sites for PMA or growth factor inducible activation (16). PMA treatment of c-fos luciferase transfected CV-1 cells resulted in an increase in normalized reporter gene activity (FIG. 1C). Like Gal4-ElkC, PMA induced c-fos luciferase activity was significantly reduced by expression of N17Ras (FIG. 1C, compare columns 2 and 4). In contrast, expression of N17Ras resulted in little inhibition of either AP-1 or NF-kB reporter gene activity in response to PMA (FIG. 1C, compare columns 6 and 8 and columns 10 and 12). These data indicate that N17Ras selectively inhibits the induction of c-fos, but not AP-1 or NF-kB reporters. The above data are surprising because N17Ras has been reported to have no effect on ERK activation induced by PMA (40-43).

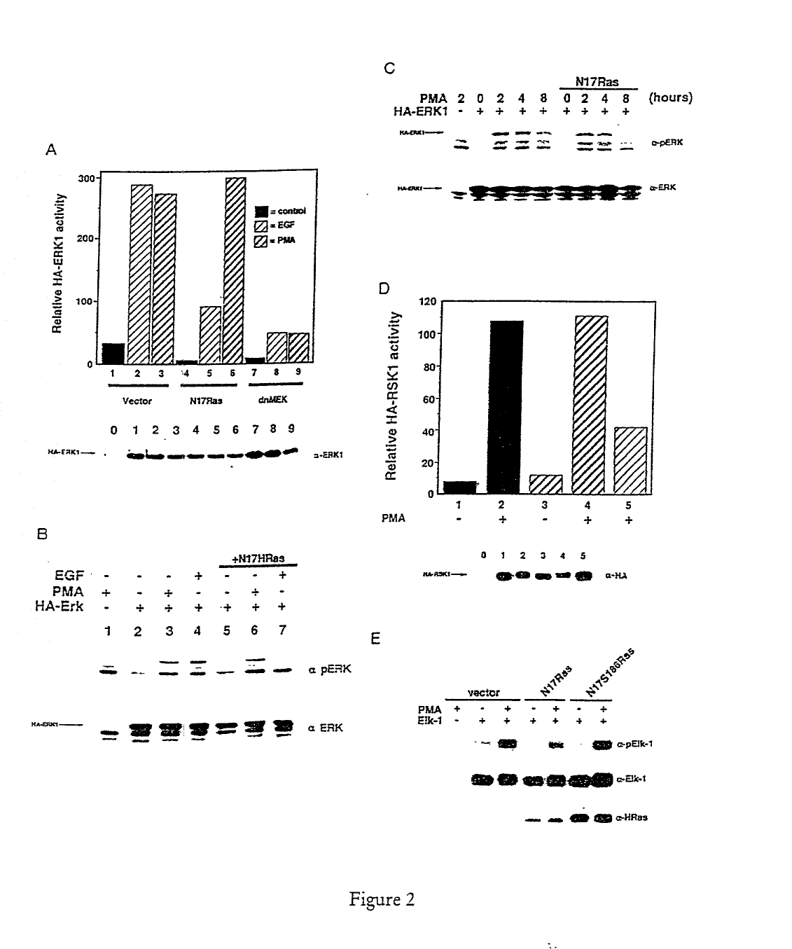

Inhibition of Elk-1 but not MAP kinase or RSK1 by N17Ras in response to PMA stimulation. A simple explanation for the strong inhibitory effect of N17Ras on TCF dependent reporter activity is that, in contrast to previous reports, N17Ras may block the activation of MAP kinase in the present cell lines in response to PMA. To test this possibility, HA-tagged ERK1 (HA-ERK1) was expressed in COS-1 cells and measured PMA or EGF induced HA-ERK1 activity in an immune-complex kinase assay using myelin basic protein (MBP) as a substrate. Expression of N17Ras, which resulted in >80% inhibition of Gal4-ELkC reporter activity, had no effect on PMA stimulated HA-ERK1 activity (FIG. 2A, compare columns 3 and 6). EGF stimulated HA-ERK1 activity, however, was inhibited by N17Ras (FIG. 2A, compare columns 2 and 5). In contrast to N17Ras, dnMEK expression blocked both PMA- and EGF-stimulated HA-ERK1 activity (FIG. 2A, columns 2 and 8).

The activation state of ERK was also determined using a phosphorylation state specific antibody (a-pERK). This antibody specifically recognizes the dual threonine/tyrosine phosphorylated ERK1 and ERK2. The ERK polyclonal antibodies (a-ERK) used were raised against recombinant ERK1 and recognize endogenous ERK1 (FIG. 2B, middle bands), transfected HA-ERK1 (FIG. 2B, upper bands) and weakly recognize ERK2 (FIG. 2B, lower bands). Expression of N17Ras had no effect on HA-ERK1 phosphorylation induced by PMA (FIG. 2B, compare lanes 3 and 6). In contrast, N17Ras strongly inhibited EGF-stimulated HA-ERK1 phosphorylation (FIG. 2B, compare lanes 4 and 7). Similar results were observed in CV-1 cells (data not shown). In addition, a time course from 0 to 8 hours was performed, monitoring HA-ERK1 phosphorylation in response to PMA in the presence or absence of N17Ras. Expression of N17Ras had little effect on HA-ERK1 phosphorylation from 0-8 hours of PMA treatment (FIG. 2C).

Together, these results support the idea that the two stimuli tested, EGF and PMA, are likely to activate ERK by distinct mechanisms, and only one of which is sensitive to inhibition by N17Ras, as is the case with EGF (40). This is consistent with previously published reports that N17Ras expression does not interfere with the activation of ERK in response to PMA treatment in COS-1 cells (40,41). However, these results do not explain the present observation that PMA-stimulated Elk-1 activity is inhibited by N17Ras, since Elk-1 is a direct target of active MAP kinases.

The effects of N17Ras on the activity of another ERK substrate, RSK1, were also examined. RSK1 is a serine/threonine kinase whose activity is enhanced upon phosphorylation by ERK in vivo (35,45). N17Ras had little effect on HA-RSK activity as determined by an immune-complex kinase assay, both under basal and PMA stimulated conditions. In contrast, expression of dominant negative MEK1 significantly abrogated PMA-stimulated HA-RSK1 activity (FIG. 2D). These observations indicate that HA-RSK activity is not inhibited by N17Ras, though ERK activation appears to be required for RSK activity.

Previous reports have established that the transactivation activity of Elk-1, as well as other TCF members, is enhanced by MAP kinase phosphorylation at specific ser/thr residues in its activation domain (13, 14, 18). In the case of Elk-1, phosphorylation at serine 383 leads to a significant enhancement of its trans-activation activity (10, 11, 15). Using an Elk-1 phosphoserine-383 specific antibody, it was observed that N17Ras reduced PMA stimulated serine 383 phosphorylation of Elk-1 (FIG. 2E). N17S186Ras, which is exclusively cytosolic (data not shown), was unable to inhibit Elk-1 phosphorylation, suggesting that membrane localization of N17Ras may be important for its ability to inhibit Elk-1 (FIG. 2E). These results support the above observation that N17Ras expression specifically reduces TCF transcription activity and further demonstrates that N17Ras inhibits Elk-1.

Effects of N17Ras or L61S186Ras on MEK-induced Elk-1 phosphorylation. Whether the inhibition of Elk-1 is a common feature of dominant interfering Ras mutants or unique to N17Ras was tested. L61S186Ras is a cytosolic GTP-bound Ras mutant that dominantly interferes with Ras signaling (23,27). Ectopic expression of L61S186Ras is likely to prevent membrane recruitment of Ras targets, such as Raf, by sequestering them from membrane targeted GTP-Ras (23,27). In contrast, N17Ras inhibits GTP-Ras formation. Therefore, L61S186 expression is thought to interfere with both wild-type and oncogenic Ras signaling (23). Therefore, it was tested whether this mutant would inhibit Elk-1 phosphorylation like N17Ras. Expression of active MEK1* resulted in a large increase in both HA-ERK1 and Elk-1 phosphorylation as detected by phospho-specific antibodies (FIG. 3A, compare lanes 1-3). N17Ras expression resulted in an inhibition of MEK1* induced Elk-1, but not HA-ERK1 phosphorylation (FIG. 3A, upper panels, compare lanes 3 and 4). In contrast, L61S186Ras had no detectable effect on either Elk-1 or HA-ERK1 phosphorylation induced by MEK1* (FIG. 3A, upper panels, compare lanes 3-5). However, the L61S186Ras used in these experiments inhibited endogenous GTP-Ras signaling since it effectively blocked EGF stimulated HA-ERK1 and Elk-1 phosphorylation (FIG. 3A, compare lanes 7 and 9). These results suggest that the inhibition of Elk-1 by N17Ras is not likely to be due to inhibition of endogenous Ras functions, and, demonstrate that the inhibition of Elk-1 phosphorylation is unique to N17Ras.

Several recent reports have demonstrated that other MAP kinase family members in addition to ERKs, are able to activate TCFs by phosphorylation (13, 14, 18, 46). Therefore, it was postulated whether N17Ras expression would inhibit Gal4-ElkC activity induced by MAP kinases other than ERK. Expression of active MEK3-DE and p38 MAP kinase resulted in a synergistic activation of the Gal4-ElkC reporter (FIG. 3B, solid bars) and co-transfection of N17Ras significantly reduced this activity (FIG. 3B, solid bars). This result is surprising since Ras function has not been directly linked to p38 activation, nor does N17Ras expression inhibit MEK3-DE induced p38 kinase activity (data not shown). As with Gal4-ElkC, MEK3-DE/p38 expression significantly elevated Gal4-ATF2 (36) activity (FIG. 3B, hatched bars) and co-transfection of N17Ras had no significant effect (FIG. 3B, hatched bars). These results suggest that the inhibitory effect of N17Ras is specific to TCFs like Elk-1, and, does not depend on activation of the ERK MAP kinase.

Negative regulation of Elk-1 by a non-interfering version of N17Ras, N17N69Ras. It was examined whether one could experimentally distinguish the two functions of N17Ras observed here, namely inhibition of Elk-1 phosphorylation versus inhibition of endogenous Ras activation. As mentioned previously, N17Ras inhibits GTP-Ras formation by targeting Ras-GEFs, like SOS. N17N69Ras is a GDP-bound form of Ras (FIG. 5C) that no longer functions as a dominant interfering mutant due to the substitution of asparagine for aspartic acid at position 69 of human HRas (20,21). Therefore, whether N17N69Ras would inhibit Elk-1 was tested. N17Ras effectively reduced EGF stimulated HA-ERK1 and Elk-1 phosphorylation (FIG. 3C, compare lanes 3 and 5). In contrast, N17N69Ras expression had little effect on EGF stimulated HA-ERK1 phosphorylation, yet inhibited Elk-1 phosphorylation (FIG. 3C, compare lanes 3, 5 and 7). These observations suggest that the ability of N17Ras to inhibit Elk-1 does not depend on its ability to inhibit endogenous Ras activation.

Inhibition of oncogenic Ras induced Elk-1 activity and focus formation by N17Ras. The previous results predict that N17Ras will inhibit Elk-1 in the presence of V12Ras. This hypothesis was tested by examining V12Ras induced SRE or c-fos reporter activity in the presence or absence of N17Ras in NIH3T3 cells. V12Ras expression elevated both SRE and c-fos reporter activity by approximately 10 fold (FIG. 4A) and co-expression of either N17, or N17N69 reduced SRE and c-fos reporter activity to near basal levels (FIG. 4A). A15Ras, another interfering Ras mutant that blocks Ras activation (19), also inhibited V12Ras induced Gal4-ElkC (FIG. 4A). The fact that expression of the MAP kinase phosphatase HVH-1 (47) also inhibited reporter activity suggests that V12Ras induced c-fos promoter requires MAP kinase activation (FIG. 4A). Activity of Gal4-TCF chimeras was also tested. V12Ras induced activation of Gal4-ElkC, Gal4-Sap1C or Gal4-Sap2C (7,9) was inhibited by expressing N17, N17N69, or A15Ras (FIG. 4B). Furthermore expression of N17, N17N69 or A15Ras inhibited V12Ras induced focus formation in NIH3T3 cells (FIG. 4C). These results suggest that certain events leading to transcription activation and cellular transformation induced by V12Ras remain sensitive to inhibition by co-expression of N17 and other GDP-bound Ras mutants.

N17Ras selectively blocks nucleotide loading of wild-type but not oncogenic Ras. These observations are not readily explained by current models of Ras function in which GTP-Ras is active and GDP-Ras is inactive, and, in which N17Ras displays a dominant negative effect by simply interfering with endogenous Ras activation. This model predicts that N17Ras should always be recessive to phenotypes elicited by V12Ras. Two simple scenarios, however, would explain these observations. First, N17Ras may interfere with the ability of V12Ras to bind GTP in vivo. Second, N17Ras may have a discreet function in signaling Elk-1 via an unknown mechanism.

Experiments were performed to directly examine the effects of N17Ras expression on the nucleotide binding status of either V12 or wild-type Ras in COS-1 cells. To this end, COS-1 cells were transfected with HA-V12Ras or HA-Ras in the presence or absence of a non-epitope tagged version of N17Ras. Serum-starved cells were labeled with 32-PO4 for 4 hours prior to stimulation and immunoprecipitation with a-HA. Nucleotides bound to the immunoprecipitated Ras were eluted and resolved by TLC. The present results indicate that HA-V12Ras was mostly complexed with GTP (FIG. 5A, left panel, lane 2) and this was not affected by co-expression of N17Ras (FIG. 5A, left panel, compare lanes 2 and 3). In contrast, wild-type HA-Ras was mainly GDP-bound and treatment of cells with EGF for two minutes resulted in a significant increase in GTP-bound HA-Ras that was completely inhibited by N17Ras (FIG. 5A, left panel, compare lanes 4-7), suggesting that N17Ras was capable of inhibiting an EGF stimulated Ras-GEF. Similarly, expression of Flag-SOS significantly increased the amount of GTP-bound HA-Ras (FIG. 5A, left panel, lane 8) and expression of N17Ras reduced this significantly (FIG. 5A, left panel, compare lanes 8 and 9). Immunoblot analysis of lysates from identically transfected cells with a-HRas and a-Flag revealed that all cDNAs were evenly expressed (FIG. 5A, right panel). Consistent with the present in vivo labeling experiments, expression of N17Ras did not alter the amount of HA-V12Ras that could bind to the Ras binding domain of c-Raf (RBD) as determined by GST pull-down and immunoblotting (FIG. 5B, left panel), though N17Ras was efficiently expressed (FIG. 5B, right panel). These data demonstrate that V12Ras is not subject to regulation by N17Ras in vivo and confirm that in COS-1 cells N17Ras can effectively interfere with wild-type Ras activation in response to EGF via Ras exchange factors, such as SOS.

N17Ras is GDP-bound in vivo. In vitro, under limiting Mg2+ and nucleotide concentrations, N17Ras binds GDP with preferential affinity, though 60-fold less effective than wild-type Ras (5,25). However, it has not been demonstrated that N17Ras is GDP-bound in vivo, though it has been predicted based on the known binding constants and intracellular Mg2+ and GDP concentrations (5,25). Therefore, the in vivo nucleotide binding specificity of various Ras mutants was determined by in vivo labeling and immunoprecipitation experiments. The inventors have obtained results indicating that greater than 90% of the nucleotides complexed with both N17 and N17N69 are GDP in vivo (FIG. 5C). Surprisingly, A15Ras, previously shown to be nucleotide free using bacterial expressed protein (19), was GDP-bound (FIG. 5C), in vivo. In contrast, the majority of nucleotides complexed with V12Ras were GTP, whereas wild type Ras is also largely GDP-bound (FIG. 5A). It is important to note that all Ras proteins tested in this assay were associated with comparable amounts of radioactivity. Therefore, assuming that all of the wild-type Ras is bound to nucleotide, N17, N17N69, and A15Ras all appear to be loaded with GDP, where as V12Ras is largely GTP-bound.

### Example 2

Animal Studies

RasV12 transformed NIH3T3 cells grown in Delbecco's modified Eagle Medium (2×10⁶ cells in 0.1 ml) are inoculated subcutaneously in the back of Bulb C athymic mice. After one week, when tumor size has reached 80-100 mg, tumors are injected with 25-125 μg of RasN17N69 protein fused with transducing peptide of HIV TAT protein as well as saline (as controls). Tumor regression is evaluated at the indicated time points, 7, 14, 21, and 28 days by calculating tumor volume (formula: 4/3 pr³ where r=(length+width)/4) and weight. The excised tumors are later utilized to determine RasN17N69 levels by Western analysis as previously described.

References

1. Lowy, D. R., and Willumsen, B. M. (1993) Annu Rev Biochem 62, 851-91

2. Katz, M. E., and McCormick, F. (1997) Curr Opin Genet Dev 7(1), 75-9

3. Land, H., Parada, L. F., and Weinberg, R. A. (1983) Nature 304(5927), 596-602

4. Mulcahy, L. S., Smith, M. R., and Stacey, D. W. (1985) Nature 313(5999), 241-3

5. Feig, L. A., and Cooper, G. M. (1988) Mol Cell Biol 8(8), 3235-43

6. Robinson, M. J., and Cobb, M. H. (1997) Curt Opin Cell Biol 9(2), 180-6

7. Treisman, R. (1994) Curr Opin Genet Dev 4(1), 96-101

8. Hill, C. S., and Treisman, R. (1995) Cell 80(2), 199-211

9. Treisman, R. (1996) Curr Opin Cell Biol 8(2), 205-15

10. Marais, R., Wynne, J., and Treisman, R. (1993) Cell 73(2), 381-93

11. Gille, H., Sharrocks, A. D., and Shaw, P. E. (1992) Nature 358(6385), 414-7

12. Gille, H., Strahl T., and Shaw, P. E. (1995) Curt Biol 5(10), 1191-200

13. Cavigelli, M., Dolfi, F., Claret, F. X., and Karin, M. (1995) Embo J 14(23), 5957-64

14. Price, M. A., Cruzalegui, F. H., and Treisman, R. (1996) Embo J 15(23), 6552-63

15. Gille, H., Kortenjann, M., Thomae, O., Moomaw, C., Slaughter, C., Cobb, M. H., and Shaw, P. E. (1995) Embo J 14(5), 951-62

16. Hill, C. S., Marais, R., John, S., Wynne, J., Dalton, S., and Treisman, R. (1993) Cell 73(2), 395-406

17. Hipskind, R. A., Rao, V. N., Mueller, C. G., Reddy, E. S., and Nordheim, A. (1991) Nature 354(6354), 531-4

18. Janknecht, R,. and Hunter, T. (1997) Embo J 16(7), 1620-7

19. Chen, S. Y., Huff, S. Y., Lai, C. C., Der, C. J., and Powers, S. (1994) Oncogene 9(9), 2691-8

20. Quilliam, L. A., Kato, K., Rabun, K. M., Hisaka, M. M., Huff, S. Y., Campbell-Burk, S., and Der, C. J. (1994) Mol Cell Biol 14(2), 1113-21

21. Quilliam, L. A., Hisaka, M. M., Zhong, S., Lowry, A., Mosteller, R. D., Han, J., Drugan, J. K., Broek, D., Campbell, S. L., and Der, C. J. (1996) J Biol Chem 271(19), 11076-82

22. Hart, M., and Powers, S. (1995) Methods Enzymol 255, 129-35

23. Stacey, D. W., Feig, L. A., and Gibbs, J. B. (1991) Mol Cell Biol 11(8), 4053-64

24. Lai, C. C., Boguski, M., Broek, D., and Powers, S. (1993) Mol Cell Biol 13(3), 1345-52

25. John, J., Rensland, H., Schlichting, I., Vetter, I., Borasio, G. D., Goody, R. S., and Wittinghofer, A. (1993) J Biol Chem 268(2), 923-9

26. Cool, R. H., Schmidt, G., Lenzen, C. U., Prinz, H., Vogt, D., and Wittinghofer, A. (1999) Mol Cell Biol 19(9), 6297-305

27. Michaeli, T., Field, J., Ballester, R., O'Neill, K., and Wigler, M. (1989) Embo J 8(10), 3039-44

28. Finney, R. E., and Bishop, J. M. (1993) Science 260(5113), 1524-7

29. Guerrero, I., Villasante, A., Corces, V., and Pellicer, A. (1985) Proc Natl Acad Sci U S A 82(23), 7810-4

30. Diamond, L. E., Guerrero, I., and Pellicer, A. (1988) Mol Cell Biol 8(5), 2233-6

31. Bremner, R., and Balmain, A. (1990) Cell 61(3), 407-17

32. Riou, G., Barrois, M., Sheng, Z. M., Duvillard, P., and Lhomme, C. (1988) Oncogene 3(3), 329-33

33. Sugimoto, T., Stewart, S., Han, M., and Guan, K. L. (1998) Embo J 17(6), 1717-27

34. Waters, S. B., Chen, D., Kao, A. W., Okada, S., Holt, K. H., and Pessin, J. E. (1996) J Biol Chem 271(30), 18224-30

35. Zhao, Y., Bjorbaek, C., and Moller, D. E. (1996) J Biol Chem 271(47), 29773-9

36. Gupta, S., Campbell, D., Derijard, B., and Davis, R. J. (1995) Science 267(5196), 389-93

37. Zheng, C. F., and Guan, K. L. (1994) J Biol Chem 269(31), 19947-52

38. Gibbs, J. B., Schaber, M. D., Schofield, T. L., Scolnick, E. M., and Sigal, I. S. (1989) Proc Natl Acad Sci USA 86(17), 6630-4

39. Clark, G. J., Cox, A. D., Graham, S. M., and Der, C. J. (1995) Methods Enzymol 255, 395-412

40. Marais, R., Light, Y., Mason, C., Paterson, H., Olson, M. F., and Marshall, C. J. (1998) Science 280(5360), 109-12

41. Ueda, Y., Hirai, S., Osada, S., Suzuki, A., Mizuno, K, and Ohno, S. (1996) J Biol Chem 271(38), 23512-9

42. de Vries-Smits, A. M., Burgering, B. M., Leevers, S. J., Marshall, C. J., and Bos, J. L. (1992) Nature 357(6379), 602-4

43. Howe, L. R., Leevers, S. J., Gomez, N., Nakielny, S., Cohen, P., and Marshall, C. J. (1992) Cell 71(2), 335-42

44. Zheng, C. F., and Guan, K. L. (1994) Embo J 13(5), 1123-31

45. Wood, K. W., Sarnecki, C., Roberts, T. M., and Blenis, J. (1992) Cell 68(6), 1041-50

46. Whitmarsh, A. J., Shore, P., Sharrocks, A. D., and Davis, R. J. (1995) Science 269(5222), 403-7

47. Zheng, C. F., and Guan, K. L. (1993) J Biol Chem 268(22), 16116-9

48. Burgering, B. M., de Vries-Smits, A. M., Medema, R. H., van Weeren, P. C., Tertoolen, L. G., and Bos, J. L. (1993) Mol Cell Biol 13(12), 7248-56

49. Park, H. O., Bi. E., Pringle, J. R., and Herskowitz, I. (1997) Proc Natl Acad Sci USA 94(9), 4463-8

50. Nehrbass, U., and Blobel, G. (1996) Science 272(5258), 120-2

51. Wotton, D., Ways, D. K., Parker, P. J., and Owen, M. J. (1993) J Biol Chem 268(24), 17975-82

All references cited throughout the specification are incorporated by reference where appropriate for appropriate teachings of additional or alternative details, features and/or technical background.

What is claimed is:

1. A method for inhibiting cellular oncogenic transformation and proliferation, or reversing cellular oncogenic transformation in Ras-mediated neoplasia comprising administering to a mammal in need thereof an effective amount of a GDP-bound Ras protein.

2. The method as claimed in claim 1, wherein the Ras protein is a non-interfering Ras.

3. The method as claimed in claim 1, wherein the Ras protein is a membrane-associated Ras.

4. The method as claimed in claim 1, wherein the Ras protein is a mutant Ras.

5. The method as claimed in claim 1, wherein the Ras protein is a wild type Ras.

6. The method as claimed in claim 4, wherein the mutant Ras protein is selected from the group consisting of RasN17N69 and RasA15N69.

7. The method as claimed in claim 1, wherein the Ras protein is administered to a cell of said mammal by induction of in situ expression of a nucleic acid molecule encoding the same.

8. The method as claimed in claim 7, wherein said nucleic acid molecule is a DNA or a RNA.

9. The method as claimed in claim 7, wherein said nucleic acid molecule is an expression construct which encodes said GDP-bound Ras protein.

10. The method as claimed in claim 9, wherein said expression construct encodes RasN17N69.

11. The method as claimed in claim 1, wherein said neoplasia is selected from the group consisting of pancreatic cancer, sporadic colorectal carcinomas, lung adenocarcinomas, thyroid cancer, and myeloid leukemia.

12. A pharmaceutical composition comprising a GDP-bound Ras protein or an expression vector expressing the same, and a pharmaceutically acceptable carrier.

13. The pharmaceutical composition as claimed in claim 10, wherein said GDP-bound Ras is a non-interfering Ras.

14. The pharmaceutical composition as claimed in claim 10, wherein said GDP-bound Ras is a membrane-associated Ras.

15. The pharmaceutical composition as claimed in claim 10, wherein said GDP-bound Ras is selected from the group consisting of RasN17N69 and A15N69.

16. A kit comprising a carrier means having in close confinement therein at least two container means, wherein a first container means contains the pharmaceutical composition of any one of the claims 10-13, and a second container means contains a chemotherapeutic agent.

17. A method of treating a Ras-mediated neoplasm comprising administering a GDP-bound Ras protein or an expression vector encoding the same together with at least one other therapy selected from the group consisting of surgical removal of part or all of the neoplasm, chemotherapy, and radiation therapy.

18. The method as claimed in claim 1, wherein the Ras protein is administered to a cell of said mammal by protein transduction.

19. A method of making a Ras mutant comprising mutating a RasN17 mutant in the switch II region.

20. The method of claim 17, wherein the switch II region comprises amino acid residues 62-70 of the Ras protein.

21. The method as claimed in claim 18, wherein at least one of the residues 62-70 is mutated to alanine or serine.

22. The method as claimed in claim 1, wherein the Ras protein is a fusion protein.
